(19) United States
(12) Patent Application Publication
(10) Pub. No.: US 2010/0184189 A1
(43) Pub. Date: Jul. 22, 2010

(54) PROTEIN FOR THE CHEMOENZYMATIC PRODUCTION OF L-THREO-HYDROXYASPARTATE

(76) Inventors: Mohamed A. Marahiel, Marburg (DE); Matthias Strieker, Marburg (DE); Lars-Oliver Essen, Marburg (DE)

Correspondence Address: CLARK & BRODY, 1700 Diagonal Road, Suite 510, Alexandria, VA, 22314, US

(21) Appl. No.: 12/595,696

(22) PCT Filed: Apr. 7, 2008
(86) PCT No.: PCT/DE08/00580

§ 371 Date: Mar. 31, 2010

(30) Foreign Application Priority Data
Apr. 13, 2007 (DE) 102007017861.3

(51) Int. Cl. C12N 9/78

(52) U.S. Cl. 435/227

(57) ABSTRACT

A simple mutant of the natural asparagine oxygenase comprises at least the amino acids 13 to 318 of the natural asparagine oxygenase AsnO. In this protein according to the present invention, comprising at least the amino acids 13-318 of AsnO D241N, the amino acid residue at position 241 of the natural asparagine oxygenase AsnO is exchanged from aspartate (D) to asparagine (N). The protein according to the present invention, comprising at least the amino acids 13-318 of AsnO D241N, is produced by means of a directed mutagenesis from the AsnO wild type, cloning of this expression plasmid into a vector, transformation of the vector plasmid construction into a host organism and expression of the recombinant protein. The protein according to the present invention is suitable for the chemoenzymatic and enantioselective production of L-threo-hydroxyaspartate from L-aspartate. The protein is substrate-specific and converts quantitatively L-aspartate into L-threo-hydroxyaspartate.

The invention at hand describes a mutated iron-(II)- and α-ketoglutarate-dependent hydroxylase. The protein according to the present invention is a mutant of the natural asparagine oxygenase AsnO (wild type), wherein the amino acid 241 of the wild type (aspartate) is exchanged with asparagine. The obtained mutant (AsnO D241N) is capable of catalyzing the quantitative synthesis of L-(−)threo-3-hydroxyaspartate from L-aspartate.

### DESCRIPTION AND INTRODUCTION TO THE GENERAL AREA OF THE INVENTION

The invention at hand concerns the areas of protein biochemistry, molecular biology and chemoenzymatic synthesis.

### STATE OF THE ART

L-glutamate plays a major role in the mammalian central nervous system (CNS), acting as a primary excitatory neurotransmitter. Hereby, L-glutamate takes part in a wide range of neuronal communications, whereby a large amount of excitatory amino acid receptors is activated which are responsible for complex signal transmissions, such as memory formation, learning processes and development, as well as the immune response to injuries. L-glutamate, however, can also over-activate these receptors and thereby also contribute to an acute or chronic damage in the CNS.

Hence, a regulation of the amount of the excitatory L-glutamate is decisive, as a lack of L-glutamate leads to a reduced signal transmission, whereas an excess causes the initiation of excitotoxic signal pathways. An excess of L-glutamate occurs, for instance, in anemia, hypoglycaemia, Huntington's disease, Alzheimer's disease, amyotrophic lateral sclerosis, tardive dyskinesias, anxiety disorders, clinical depressions, schizophrenia, epilepsy, astrocytomas and some liver diseases.

The major part of the glutamate transport in the CNS is mediated by high-affinity and sodium-dependent EAA transporters (“excitatory amino acid transporter”).

It was shown that the EAA analog L-threo-hydroxaspartate inhibits the function of the glutamate transporters (R J Bridges, M P Kavanaugh, A R Chamberlin: A pharmacological review of competitive inhibitors and substrates of high-affinity, sodium-dependent glutamate transport in the central nervous system. Curr Pharm Des 1999, 5, 363-379). The benzylated derivative (L-TBOA) also comprises this inhibitor effect (K Shimamoto, B Lebrun, Y Yasuda-Kamatani, M Sakaitani, Y Shigeri, N Yumoto, T Nakajima: DL-threo-beta-benzyloxyaspartate, a potent blocker of excitatory amino acid transporters. Mol Pharmacol 1998, 53, 195-201 and K Shimamoto, Y Shigeri, Y Yasuda-Kamatani, B Lebrun, N Yumoto, T Nakajima: Syntheses of optically pure beta-hydroxyaspartate derivatives as glutamate transporter blockers. Bioorg Med Chem Lett 2000, 10, 2407-2410).

The synthesis of a racemic mixture of DL-threo-hydroxyaspartate and its erythreo form was described for the first time in 1921 by Dakin. This synthesis route starts with fumarate or malate as starting material. The unsaturated acid is converted to chloromalate and subsequently aminated to hydroxyaspartate (HD Dakin: The synthesis of inactive para- and anti-hydroxyaspartic acids (aminomalic acids). J Biol Chem 1921, 48, 273-291).

The second known synthesis route is described in detail for β-methyl-β-hydroxyaspartate and comprises a condensation of the aldol type between copper glycinate and a carbonyl compound (L Benoiton, S M Birnbaum, M Winitz, J P Greenstein: The enzymatic route of beta-methylaspartic acid with acylase II. Arch Biochem Biophys 1959, 81, 434-438). This synthesis route, however, leads to a complex diastereomeric mixture.

The synthesis of optically pure L-TBOA, the most efficient inhibitor of the glutamate transporters, starts with (R)-Garner-aldehyde, comprises seven synthesis steps and is time- and cost-intensive (K Shimamoto, Y Shigeri, Y Yasuda-Kamatani, B Lebrun, N Yumoto, T Nakajima: Syntheses of optically pure beta-hydroxyaspartate derivatives as glutamate transporter blockers. Bioorg Med Chem Lett 2000, 10, 2407-2410).

Methods for the production of L-threo-hydroxyaspartate known to date are time-consuming, cost-intensive and do not yield an enantiomerically pure product. A method for the enantioselective synthesis of L-threo-hydroxyaspartate (L-THA) would be, however, desirable, as L-TBOA can easily be produced from L-THA. The invention at hand provides a novel protein which enables the chemoenzymatic and enantiomerically pure production of L-threo-hydroxyaspartate from L-aspartate.

### AIM

The aim of the invention at hand is to provide a protein which catalyzes the chemoenzymatic and enantioselective synthesis of L-threo-hydroxyaspartate from L-aspartate.

### ACHIEVEMENT OF THIS AIM

This aim is achieved according to the present invention through a protein comprising at least the amino acids 13-318 of AsnO D241N, wherein
AsnO D241N represents a simple mutant of the natural asparagine oxygenase,
the amino acid residue at position 241 of the asparagine oxygenase AsnO is exchanged from aspartate (D) to asparagine (N)
under simple mutant, it is understood that compared to the natural protein exactly one amino acid is exchanged.

The natural protein AsnO (asparagine oxygenase) is part of the CDA biosynthesis gene cluster in Streptomyces coelicor, wherein CDA means “calcium-dependent antibiotic”. AsnO is a Fe²⁺ and α-ketoglutarate-dependent hydroxylase which exclusively catalyzes the production of L-threo-hydroxyaspartate from L-asparagine. L-threo-hydroxyaspartate acts in vivo as a building block for non-ribosomally produced CDA. During the catalytic cycle, this class of enzymes couples the oxidative decomposition of α-ketoglutarate to succinate and CO₂ with the hydroxylation of the substrate (L-asparagine).

In the wild type of AsnO, thereby the side chain of the amino acid residue Asp-241 binds to the NH₂ group of the carboxamide group of L-Asn.

Surprisingly, it has been found that a directed mutagenesis of Asp-241 to Asn-241 (D241N) results in a binding site for the carboxyl group of an aspartate side chain. By means of this directed mutagenesis, the substrate specificity changes from asparagine to aspartate, and the mutated protein converts aspartate chemoenzymatically to L-threo-hydroxyaspartate in the presence of Fe²⁺ and α-ketoglutarate (α-KG). The reaction occurs in a quantitative and enantioselective manner according to the following scheme:

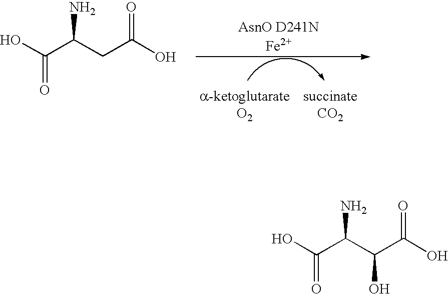

The protein mutated in this way, is hereinafter called AsnO D241N. According to the present invention, AsnO D241N is called “simple mutant” of the wild type AsnO. Hereby, “simple mutant” means that in AsnO D241N exactly one amino acid (here: amino acid 241) is modified as compared to the wild type.

Surprisingly, it has been found that a protein which comprises at least the amino acid sequence of the AsnO D241N from the first to the last secondary structure element converts L-aspartate into the corresponding L-threo-hydroxyaspartate. Hereby, “from the first to the last secondary structure element” means that at least the sequence of amino acid D13 (aspartate) to M318 (methionine) of AsnO D241N has to be present.

In a particularly preferred embodiment the protein, which catalyzes the conversion from L-Asp to L-threo-hydroxyaspartate, comprises the amino acids A2 (alanine) to A333 (alanine) of AsnO D241N.

According to the present invention, a protein comprising at least the amino acids 13-318 of AsnO D241N is produced carrying out, firstly, a directed mutagenesis of the gene of the AsnO wild type; subsequently the cloning of this gene into a vector occurs and finally a host organism is transformed with the expression vector and the recombinant protein is expressed.

According to the present invention, during the directed mutagenesis the codon GAC (bases 721-723), which codes in the wild type for Asp-241, is replaced by a codon which codes for Asn. It is known to persons skilled in the art that the codons AAC and AAT code for asparagine. According to the present invention, the directed mutagenesis can consist of the replacement of GAC with AAC or of GAC with AAT.

In a preferred embodiment, GAC is replaced with AAC during the directed mutagenesis. It is known to persons skilled in the art that the directed mutagenesis can be carried out through the selection of suitable oligonucleotide primers with corresponding sites of mutagenesis. He is able to apply this knowledge without leaving the scope of protection of the patent claims.

Analogously to the definition of the term “simple mutant” on protein level, “simple mutant” on nucleic acid level means that exactly one codon (here: bases 721-723) is modified as compared to the wild type.

It is known to persons skilled in the art how to clone oligonucleotides into vectors. A suitable vector is, for instance, pQTEV.

Bacterial strains, for instance Eschericia coli, are known to the person skilled in the art to be suitable as host organism, which can be transformed with the expression vector and can then express the recombinant protein.

The protein according to the present invention, comprising at least the amino acids 13-318 of AsnO D241N, is produced by a method comprising the following steps:
a) production of a oligonucleotide (gene) which comprises at least the bases 37-954 of the DNA sequence of AsnO,
b) directed mutagenesis of this gene, wherein the aspartate codon of the bases 721-723 of AsnO is replaced with an asparagine codon,
c) cloning of this gene in an expression vector,
d) transformation of a host organism with the expression vector and expression of the recombinant protein.

In a preferred embodiment, the expression plasmid is produced in the form of a His fusion plasmid, which leads to the expression of a protein according to the present invention with an N-terminal His tag. Hereby, tags with 5 to 8 histidine residues following one another are preferred. It is known to persons skilled in the art that it is advantageous to purify such His tag fusion proteins by means of affinity chromatography using a Ni-chelate column.

The protein according to the present invention is suitable for being used for the production of L-threo-hydroxyaspartate. For that purpose, L-aspartate is incubated in the presence of the protein according to the present invention and the cofactor Fe²⁺— as well as the cosubstrate α-ketoglutarate. Hereby, a non-activated β-methylene group (here: the β-CH₂ group of aspartate) is hydroxylated enzymatically. This enzymatic hydroxylation is particularly advantageous, as it is generally difficult to access the non-activated β-CH₂ groups using classical chemical ways. Furthermore, “purely chemical”, not enzymatically, catalyzed reactions of β-CH₂ groups of amino acids generally yield enantiomeric or diastereomeric mixtures. The chemoenzymatic production of L-threo-hydroxyaspartate with the help of the protein according to the present invention occurs, however, in an enantioselective and substrate-specific manner, as AsnO D241N hydroxylates exclusively L-aspartate and exclusively L-threo-hydroxyaspartate is produced.

### EMBODIMENTS

### Practical Embodiment 1

### Production of the Expression Plasmid and Directed Mutagenesis

A directed mutagenesis of the AsnO wild type is carried out, wherein the codon for Asp-241 of the wild type is modified to Asn-241. The used AsnO is part of the CDA biosynthesis gene cluster in Streptomyces coelicor, wherein CDA means “calcium-dependent antibiotic”. SEQ ID No: 1 shows the DNA sequence of the AsnO wild type.

The recombinant gene fragments are amplified by means of polymerase chain reaction from chromosomal DNA of Streptomyces coelicolor A3(2) (DSM 40783) using the Phusion Polymerase (Finnzymes). According to the manufacturer's instructions for template DNA with high GC concentration (S. coelicolor, 74%) the dNTP concentration is increased to 20 mM.

For the execution of the directed mutagenesis the QuickChange II Site-directed Mutagenesis Kit (Stratagene) is used according to manufacturer's instructions. The synthetic oligonucleotide primers (Operon)

(SEQ ID No: 5)

5′-CCCCGACCTGCGGGTGAACCTGGCGGCCACCGAGC-3′
and

(SEQ ID No: 6)

5′-GCTCGGTGGCCGCCAGGTTCAC CCGCAGGTCGGGG-3′

are used; the site of mutagenesis is underlined.

The identity of the mutated plasmid produced in this way is confirmed by means of DNA dideoxy sequencing.

SEQ ID No: 3 shows the DNA sequence of AsnO D241N; SEQ ID No: 11 shows the DNA sequence of the corresponding His₇ fusion (1.074 kb).

### Practical Embodiment 2

### Production of the Recombinant Enzyme

The mutated AsnO gene is cloned into the Bam HI sites and Not I sites of the pQTEV vector (SEQ ID No: 17, GenBank accession number AY_{—}243506). This pQE30-based (Qiagen) cloning product is used for the transformation of E. coli BL21 (DE3) (Novagen). The transformed cells are cultivated at 37° C. up to an optical density of 0.5 (λ=600 nm), induced with 1 mM isopropyl-β-D-thiogalactopyranoside and harvested at 30° C. after further 3 h. The recombinant proteins are purified by means of Ni-NTA affinity chromatography (Amersham Pharmacia Biotech). A 12% SDS-PAGE gel to control the purification is shown in FIG. 2.

The lanes with purified protein are cut from the gel, combined and subjected to buffer exchange in 25 mM HEPES, 50 mM NaCl, pH 7.0, using Hi-Trap desalting columns (Amersham Pharmacia Biotech). The concentration of the purified protein is determined spectrophotometrically at 280 nm using calculated extinction coefficients. After flash freezing in liquid nitrogen, the purified protein is stored until further use at −80° C.

SEQ ID No: 2 shows the amino acid sequence of the AsnO wild type.

SEQ ID No: 12 shows the amino acid sequence of the AsnO D241N His₇ tag;

SEQ ID No: 10 shows the amino acid sequence of AsnO D241N without cloning artifacts: these are the His7 tag, a short linker region and the “tobacco etch virus protease” recognition site. The amino acid 1 of the AsnO wild type is methionine. Using the pQTEV vector the methionine codon of the AsnO wild type is converted into a serine codon in such a way that in SEQ ID No: 8 and SEQ ID No: 10 the first amino acid of the AsnO D241N is serine and not methionine.

### Practical Embodiment 3

### Activity of the AsnO D241N Mutant

In order to evaluate the activity of the AsnO D241N mutant, the purified enzyme

AsnO D241N (40 μM) is incubated with 1.5 mM L-Asp, 1.0 mM (NH₄)₂Fe(SO₄)₂ as source for the iron cofactor and 1.0 mM α-ketoglutarate (cosubstrate) at different temperatures between 16° C. and 37° C. during 16 h. The reaction is monitored by means of HPLC MS, by scanning the masses for L-Asp ([M+H]⁺=134.5 Da) and its hydroxylated form ([M+H]⁺=150.04 Da). The incubation of L-aspartate with AsnO D241N leads to the quantitative conversion thereof to L-threo-3-hydroxyaspartate at 16° C. (FIG. 3a). In the control reaction, in which no enzyme is added, only L-Asp can be detected (FIG. 3b). Commercially available L-Asp (Bachem) as well as L-threo-3-hydroxyaspartate (Tocris Bioscience) are used as standards for the comparison of the retention times with HPLC.

### Practical Embodiment 4

### Evaluation of the Kinetic Parameters

For the evaluation of the kinetic parameters at different concentrations of the substrate L-Asp (50 μM to 2 mM), the latter was incubated as described under practical embodiment 3 with AsnO D241N (40 μM), 1.0 mM (NH₄)₂Fe(SO₄)₂ as source of the iron cofactor and 1.0 mM α-ketoglutarate (cosubstrate) and the enzyme assay was stopped at different points in time by adding nonafluoropentane acid. Furthermore, nonaflluoropentane acid functions as ion pairing reagent in the case of HPLC analysis.

The graphic plot of the initial speeds against the substrate concentrations shows that it is a Michaelis-Menten kinetics (FIG. 4a).

The kinetic parameters of AsnO D241N are subsequently determined by means of a Lineweaver-Burk equation (FIG. 4b).

AsnO D241N results in K_{M}=0.457±0.031 mM with k_{cat}=1.0±0.1 min⁻¹ for L-Asp. The catalytic efficiency is k_{cat}/Kₘ=2.2±0.4 min⁻¹*mM⁻¹. In comparison to that, the wild type AsnO comprises an almost identical Kₘ value of 0.478±0.067 mM for L-Asn, but possesses a 300 times higher k_{cat} (298±19 min⁻¹).

TABLE 1
substrate specificity of AsnO D241N

| | | [M + H]⁺ | | |
|---|---|---|---|---|
| | [M + H]⁺ | hydroxylation | [M + H]+ | |
| Amino acid | calculated | product | found | hydroxylation |
| L-Asp | 134.0 | 150.0 | 150.0 | yes |
| D-Asp | 134.0 | 150.0 | 134.1 | no |
| L-Asn | 133.1 | 149.1 | 133.0 | no |
| L-Gln | 147.1 | 163.1 | 147.2 | no |
| L-Glu | 148.1 | 164.1 | 148.2 | no |
| L-Ile | 132.1 | 148.1 | 132.2 | no |
| L-Phe | 166.1 | 182.1 | 166.1 | no |
| L-Trp | 205.1 | 221.1 | 205.1 | no |
| L-Val | 118.1 | 134.1 | 118.0 | no |

### Practical Embodiment 5

### Substrate Specificity of the Mutated Protein AsnO D241N

The specificity of AsnO D241N for the conversion of L-Asp in L-threo-3-hydroxyaspartate is examined by incubating this mutated protein during 16 h at 16° C. with the amino acids (1.5 mM) listed in Table 1, the cofactor (NH₄)₂Fe(SO₄)₂ (1 mM), the cosubstrate α-ketoglutarate (4 mM) and 20 μg catalase in 250 to 1 mL of a 50 mM HEPES buffer (pH 7.5). The catalase is added in order to quench reactive oxygen species and to prevent hereby the autoxidation of the enzyme. Controls are carried out in the absence of AsnO D241N.

The reaction is stopped by the addition of 50-200 μL of a 4% solution (V/V) of nonafluoropentane acid and subsequently the hydroxylation of the amino acid substrate to the respectively corresponding hydroxy amino acid is examined by means of reversed phase HPLC/MS. Column: Hypercarb (Thermo Electron Corporation, pore diameters of 250 Å, particle size of 5 μM, 100% carbon). Mobile phases: A=20 mM aqueous nonafluoropentane acid, B=acetonitrile. Gradient: 0-10% B in 12 min, flow rate 0.2 mL/min at 17.5° C.

The reaction product of the incubation of the respective amino acid with AsnO D241N is carried out by means of highest resolution MS with an API Qstar Pulsar I (Applied Biosystems).

### FIGURE LEGENDS

FIG. 1 Crystal structure of AsnO with bound L-threo-J3-hydroxyasparagine and succinate (PDB accession code: 2OG7). The mutant Asp-241 to Asn-241 stabilizes Asp within the binding site and changes hereby the substrate specificity from L-Asn to L-Asp.

FIG. 2 FIG. 2 shows a 12% SDS-PAGE of the purification of AsnO D241N by means of
Ni-NTA affinity chromatography.
M protein marker (Fermentas, PageRuler)
B.I. before induction
A.I. after induction
T1 first flow-through fraction of the raw lysate
T2 last flow-through fraction of the raw lysate
Lanes 5-13: elution fractions of the Ni-NTA affinity chromatography elution occurs by competitive replacement of the His₇ tag by an imidazole solution (250 mM, buffered with 50 mM HEPES and 100 mM NaCl, pH=7.5).

FIG. 3 Quantitative conversion of L-Asp in L-threo-3-hydroxyaspartate; determination by means of HPLC-MS

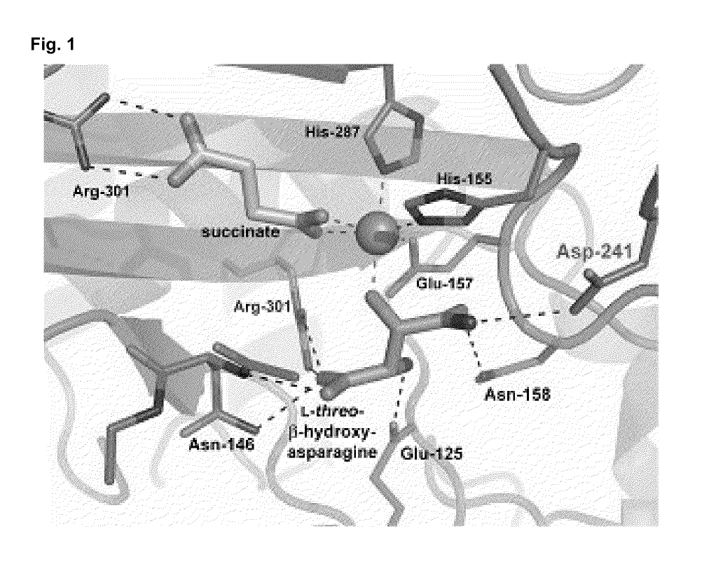

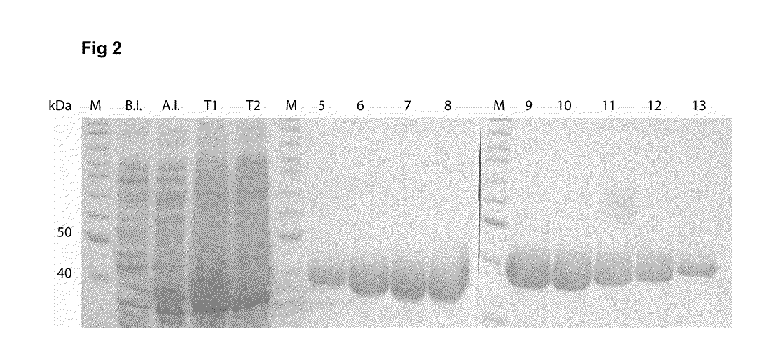

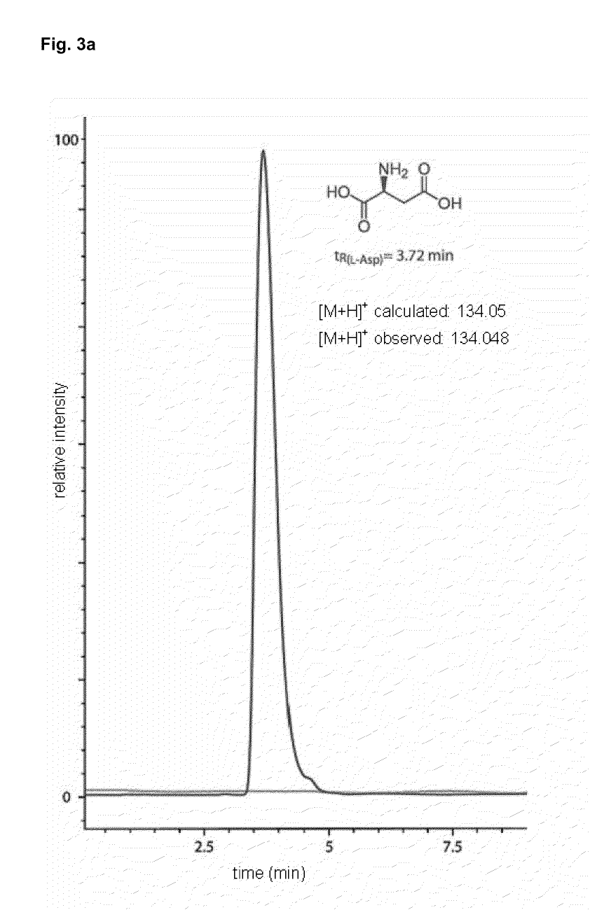

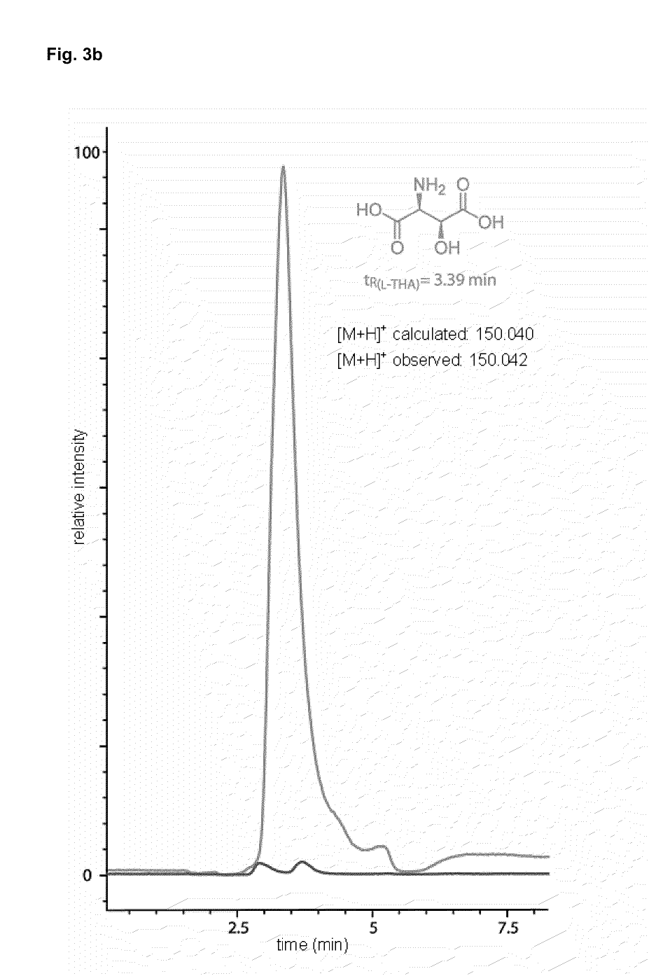

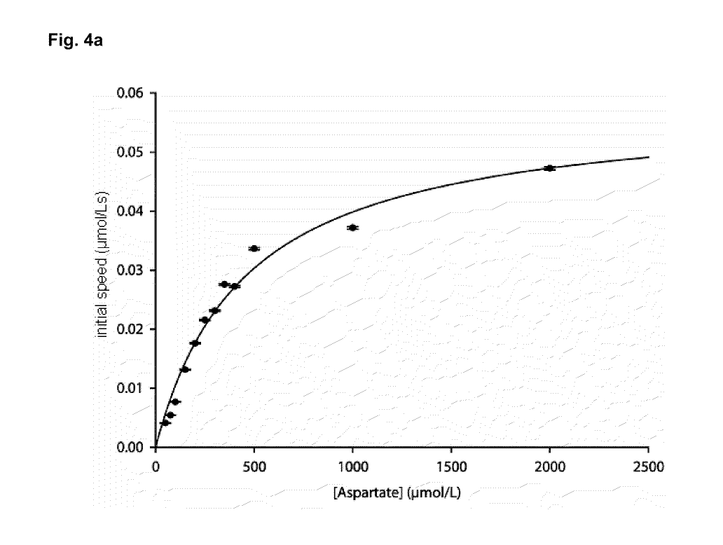

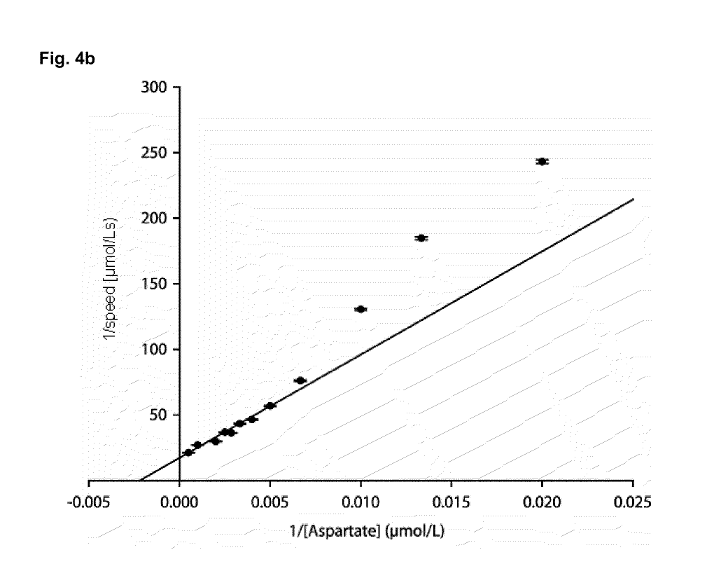

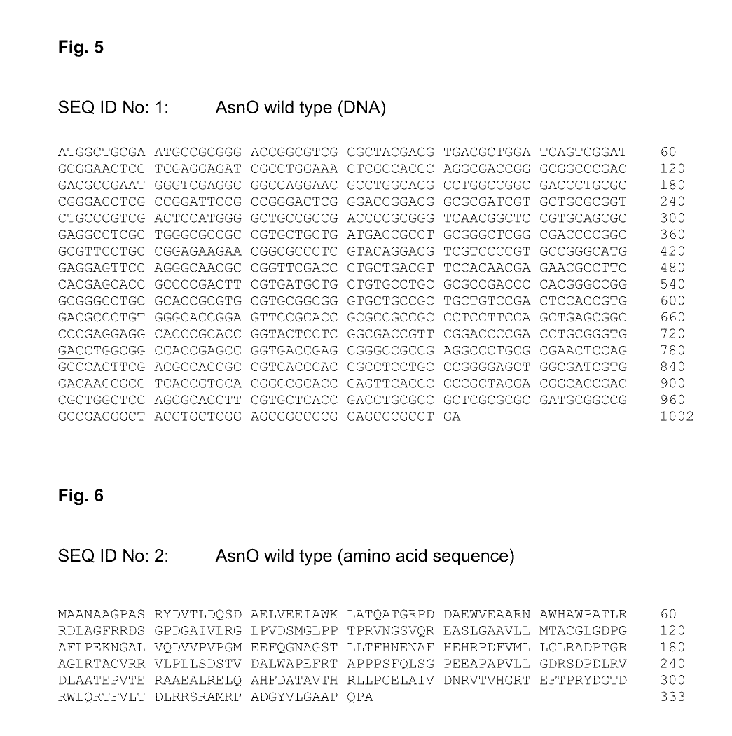

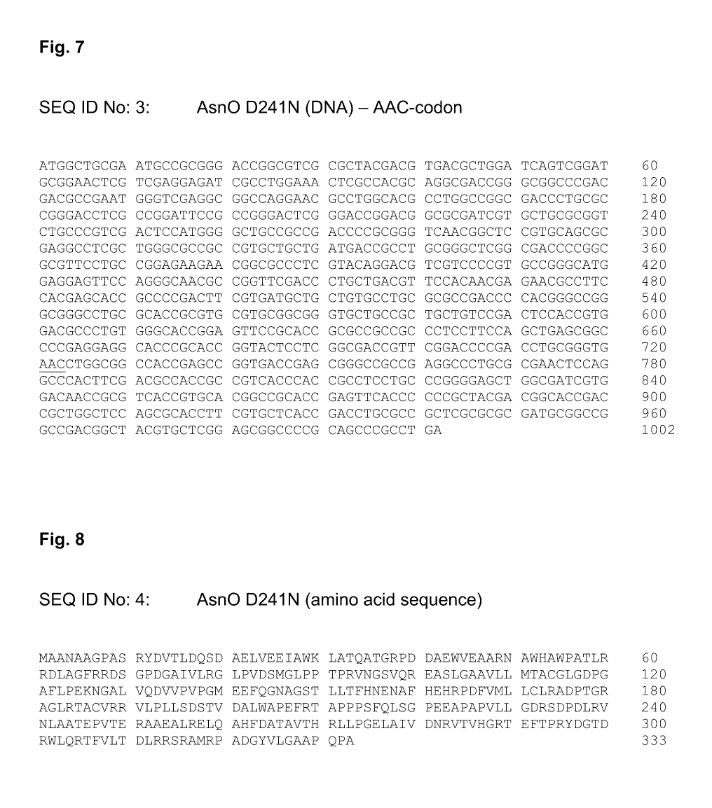

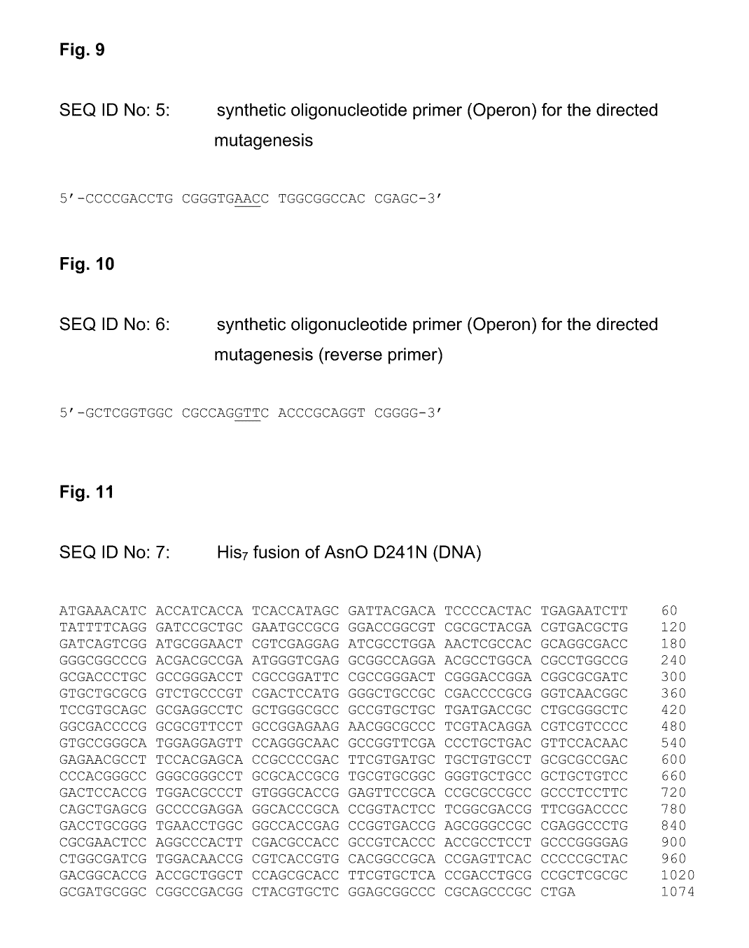

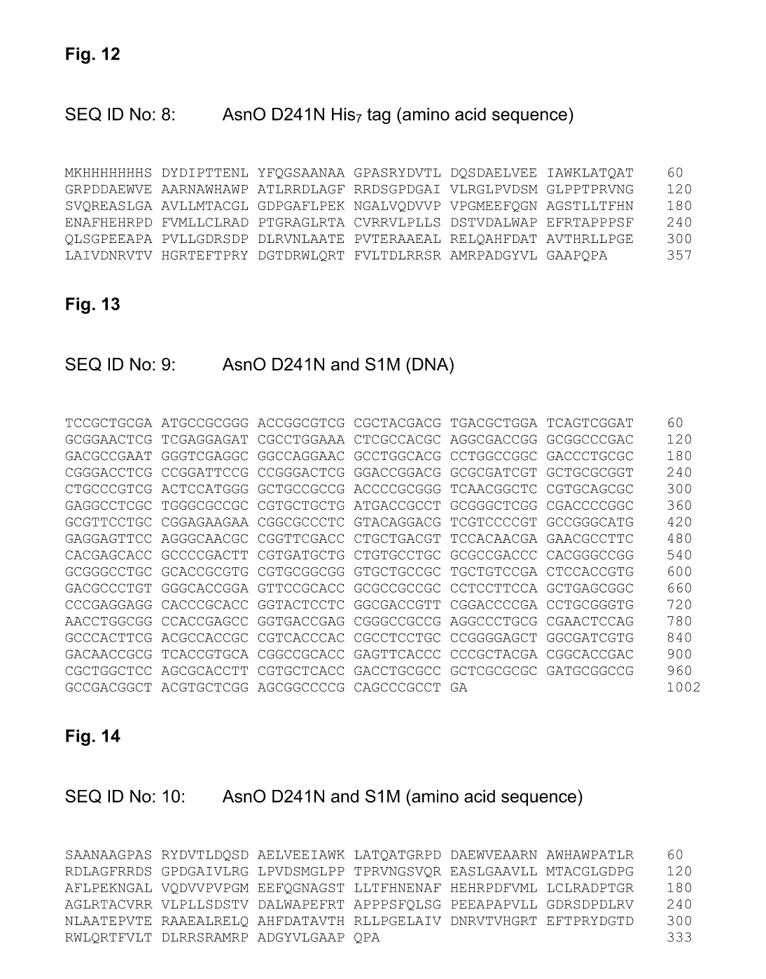

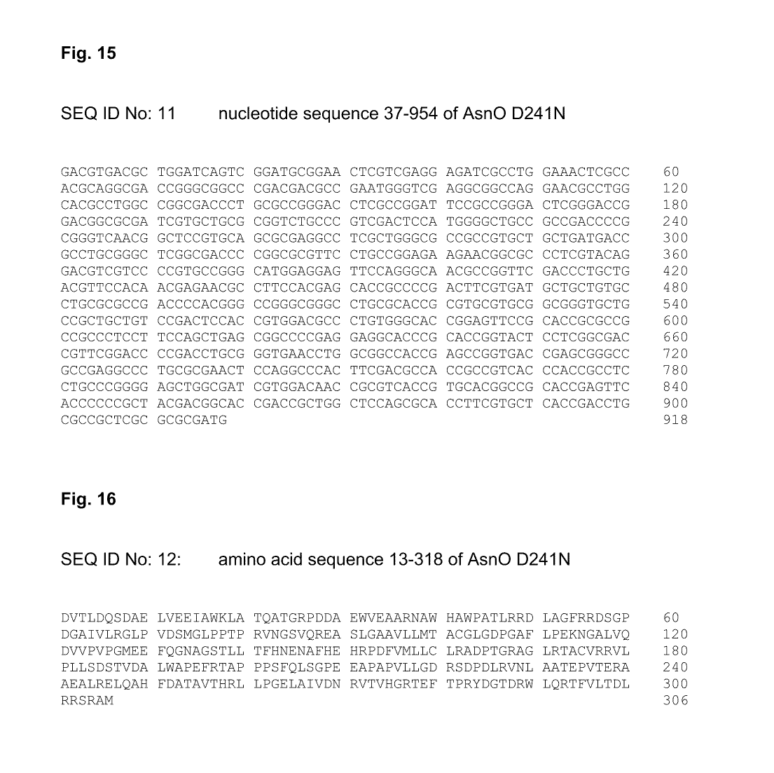

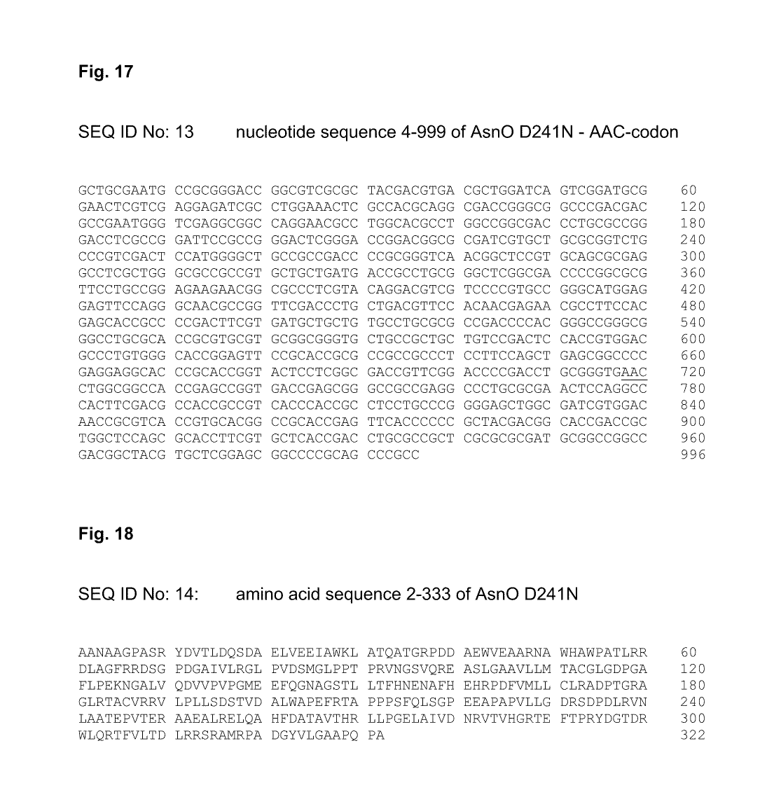

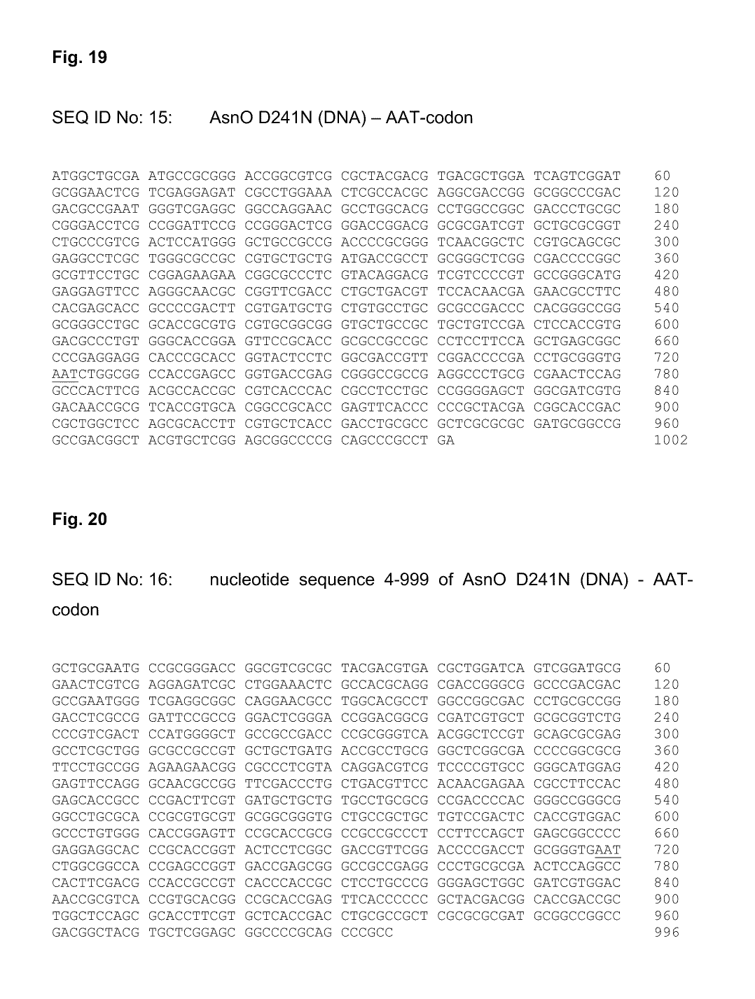

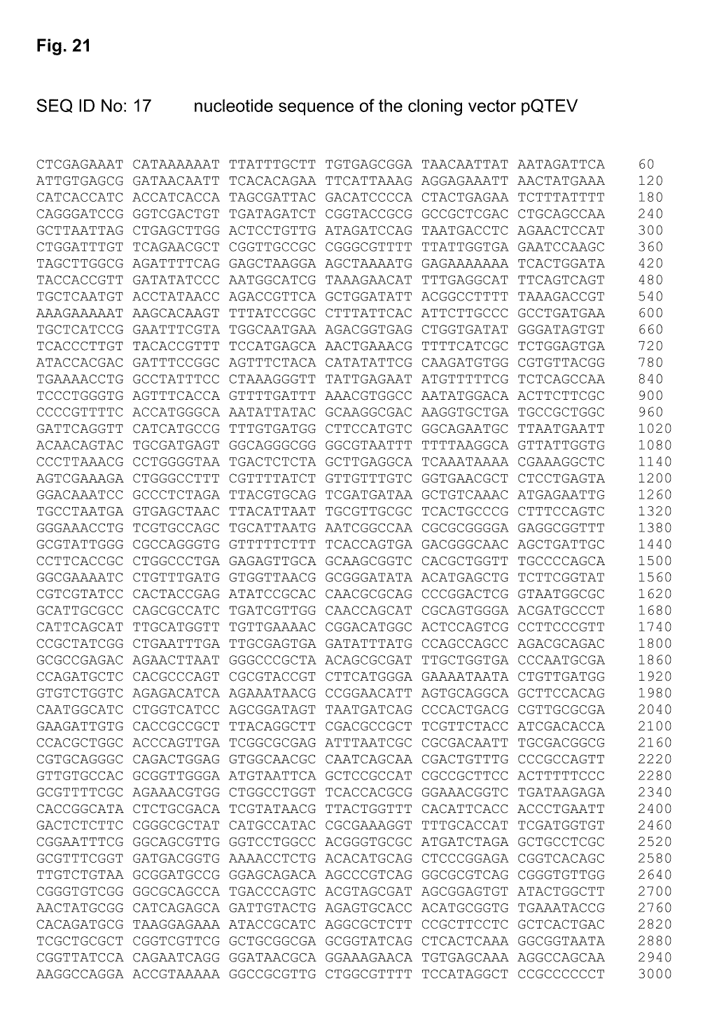

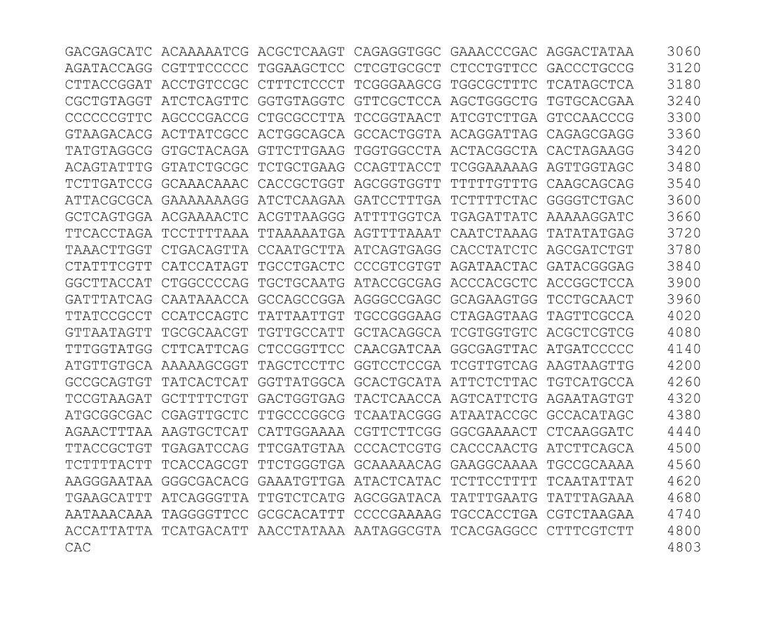

Column: Hypercarb (Thermo Electron Corporation, pore diameters of 250 Å, particle size of 5 μM, 100% carbon). Mobile phases: A=20 mM aqueous nonafluoropentane acid, B=acetonitrile Gradient: 0-10% B in 12 min, flow rate 0.2 mL 1 min at 17.5° C.
FIG. 3a: negative control, no addition of enzyme (=AsnO D241N)
   Only L-Asp is detected.
   Retention time: 3.72 min;
   M_{ber}=134.045, M_{gef}=134.048
FIG. 3b: incubation of L-Asp with AsnO D241N
   After 16 h of incubation at 16° C. only
   L-threo-3-hydroxyaspartate is detected.
   Retention time 3.39 min;
   M_{ber}=150.040, M_{gef}=150.042

The detection occurs by means of high resolution MS.

FIG. 4 4a: Michaelis-Menten diagram for the conversion of L-Asp in L-threo-3-hydroxyaspartate through AsnO D241N

4b: Lineweaver-Burke diagram for the conversion of L-Asp into L-threo-3-hydroxyaspartate through AsnO D241N

FIG. 5 SEQ ID No: 1 AsnO wild type (DNA)

GenBank accession number of the AsnO wild type (DNA): NC_{—}003888
Gen: complement (3587687 . . . 3588688)

### Locus_tag=“5003236”

synonym: “SCE29.05c”

FIG. 6 SEQ ID No: 2 AsnO wild type (amino acid sequence)

GenBank accession number of the AsnO wild type (protein): NP_{—}627448

FIG. 7 SEQ ID No: 3 AsnO D241N (DNA)-AAC-codon

bases 721-723: AAC instead of GAC in the wild type of the AsnO

FIG. 8 SEQ ID No: 4 AsnO D241N (amino acid sequence)

Mutant of the natural asparagine oxygenase AsnO; Asp-241 has been replaced by Asn-241

FIG. 9 SEQ ID No: 5 synthetic oligonucleotide primer (Operon) for the directed mutagenesis of the AsnO wild type
Exchange of the GAC (wild type) for AAC (AsnO D241N)

FIG. 10 SEQ ID No: 6 synthetic oligonucleotide primer (Operon) for the directed mutagenesis of the AsnO wild type (reverse primer)
Reverse primer: exchange of GTC (wild type) for GTT (AsnO D241N)

FIG. 11 SEQ ID No: 7 His₇ fusion of AsnO D241N (DNA)
(His, fusion insert for the expression of AsnO D241N)

FIG. 12 SEQ ID No: 8 AsnO D241N His₇ tag (amino acid sequence)
Sequence of the expressed protein after purification by means of Ni-NTA affinity chromatography

FIG. 13 SEQ ID No: 9 AsnO D241N and M1S (DNA)

Base sequence of AsnO D241N without cloning artifacts; these are the His₇ tag, a short linker region and the “Tobacco Etch Virus Protease” recognition site.

FIG. 14 SEQ ID No: 10 AsnO D241N and M1S (amino acid sequence)

Amino acid sequence of AsnO D241N without cloning artifacts; these are the His₇ tag, a short linker region and the “tobacco etch virus protease” recognition site.

FIG. 15 SEQ ID No: 11 nucleotide sequence 37-954 of AsnO D241N

FIG. 16 SEQ ID No: 12 amino acid sequence 13-318 of AsnO D241N

FIG. 17 SEQ ID No: 13 nucleotide sequence 4-999 of AsnO D241N

FIG. 18 SEQ ID No: 14 amino acid sequence 2-333 of AsnO D241N

FIG. 19 SEQ ID No: 15 AsnO D241N (DNA)-AAT-codon

Bases 721-723: AAT instead of GAC in the wild type of the AsnO

FIG. 20 SEQ ID No: 16 nucleotide sequence 4-999 of AsnO D241N-AAT-codon

FIG. 21 SEQ ID No: 17 nucleotide sequence of the cloning vector pQTEV

GenBank accession number: AY 243506

1. Protein comprising at least the amino acids 13-318 of AsnO D241N, wherein
AsnO D241N represents a simple mutant of the natural asparagine oxygenase,
the amino acid residue at position 241 of the asparagine oxygenase AsnO is exchanged from aspartate (D) to asparagine (N)
under simple mutant, it is understood that compared to the natural protein exactly one amino acid is exchanged.

2. Protein according to claim 1, comprising an amino acid sequence according SEQ ID No: 12.

3. Protein according to claim 1, comprising an amino acid sequence according SEQ ID No: 14.

4. Method for the production of a protein according to claim 1, comprising the following steps:
a) production of an oligonucleotide (gene) which comprises at least the bases 37-954 of the DNA sequence of AsnO,
b) directed mutagenesis of this gene, wherein the aspartate codon of the bases 721-723 of AsnO is replaced with an asparagine codon,
c) cloning of this gene in an expression vector,
d) transformation of a host organism with the expression vector and expression of the recombinant protein.

5. Method according to claim 4, wherein the aspartate codon of the bases 721-723 of AsnO is exchanged with the asparagine codon AAC during the directed mutagenesis.

6. Method according to claim 4, wherein the aspartate codon of the bases 721-723 of AsnO is exchanged with the asparagine codon AAT during the directed mutagenesis.

7. Method according to claim 4, wherein the expression plasmid is a His fusion plasmid.

8. Method according to claim 4, wherein pQTEV is used as the vector.

9. Method according to one of the claims 4 to 8 claim 4, wherein Eschericia coli is used as the host organism.

10. Protein which is suitable to be obtained through one method according to claim 4.

11. Use of a protein according to claim 1 for the chemoenzymatic and enantioselective production of L-threo-hydroxyaspartate.


SEQUENCE LISTING (17 sequences)

SEQ ID NO: 1 - DNA, 1002 bp, Streptomyces coelicolor
  Gene complement (3587687..3588688) Locus_tag=SCO3236 Synonym SCE29.05c
atggctgcgaatgccgcgggaccggcgtcgcgctacgacgtgacgctggatcagtcggatgcggaactcgtcgaggagatcgcctggaaactcgccacgcaggcgaccgggcggcccgacgacgccgaatgggtcgaggcggccaggaacgcctggcacgcctggccggcgaccctgcgccgggacctcgccggattccgccgggactcgggaccggacggcgcgatcgtgctgcgcggtctgcccgtcgactccatggggctgccgccgaccccgcgggtcaacggctccgtgcagcgcgaggcctcgctgggcgccgccgtgctgctgatgaccgcctgcgggctcggcgaccccggcgcgttcctgccggagaagaacggcgccctcgtacaggacgtcgtccccgtgccgggcatggaggagttccagggcaacgccggttcgaccctgctgacgttccacaacgagaacgccttccacgagcaccgccccgacttcgtgatgctgctgtgcctgcgcgccgaccccacgggccgggcgggcctgcgcaccgcgtgcgtgcggcgggtgctgccgctgctgtccgactccaccgtggacgccctgtgggcaccggagttccgcaccgcgccgccgccctccttccagctgagcggccccgaggaggcacccgcaccggtactcctcggcgaccgttcggaccccgacctgcgggtggacctggcggccaccgagccggtgaccgagcgggccgccgaggccctgcgcgaactccaggcccacttcgacgccaccgccgtcacccaccgcctcctgcccggggagctggcgatcgtggacaaccgcgtcaccgtgcacggccgcaccgagttcaccccccgctacgacggcaccgaccgctggctccagcgcaccttcgtgctcaccgacctgcgccgctcgcgcgcgatgcggccggccgacggctacgtgctcggagcggccccgcagcccgcctga

SEQ ID NO: 2 - protein, 333 aa, Streptomyces coelicolor
  natural asparagine-oxygenase from Streptomyces coelicolor
MAANAAGPASRYDVTLDQSDAELVEEIAWKLATQATGRPDDAEWVEAARNAWHAWPATLRRDLAGFRRDSGPDGAIVLRGLPVDSMGLPPTPRVNGSVQREASLGAAVLLMTACGLGDPGAFLPEKNGALVQDVVPVPGMEEFQGNAGSTLLTFHNENAFHEHRPDFVMLLCLRADPTGRAGLRTACVRRVLPLLSDSTVDALWAPEFRTAPPPSFQLSGPEEAPAPVLLGDRSDPDLRVDLAATEPVTERAAEALRELQAHFDATAVTHRLLPGELAIVDNRVTVHGRTEFTPRYDGTDRWLQRTFVLTDLRRSRAMRPADGYVLGAAPQPA

SEQ ID NO: 3 - DNA, 1002 bp, Streptomyces coelicolor
  site-directed DNA-mutagenesis of natural asparagine oxygenase AsnO Codon 721-723 (GAC in wildtype for Asp) is replaced by AAC (for Asn)
atggctgcgaatgccgcgggaccggcgtcgcgctacgacgtgacgctggatcagtcggatgcggaactcgtcgaggagatcgcctggaaactcgccacgcaggcgaccgggcggcccgacgacgccgaatgggtcgaggcggccaggaacgcctggcacgcctggccggcgaccctgcgccgggacctcgccggattccgccgggactcgggaccggacggcgcgatcgtgctgcgcggtctgcccgtcgactccatggggctgccgccgaccccgcgggtcaacggctccgtgcagcgcgaggcctcgctgggcgccgccgtgctgctgatgaccgcctgcgggctcggcgaccccggcgcgttcctgccggagaagaacggcgccctcgtacaggacgtcgtccccgtgccgggcatggaggagttccagggcaacgccggttcgaccctgctgacgttccacaacgagaacgccttccacgagcaccgccccgacttcgtgatgctgctgtgcctgcgcgccgaccccacgggccgggcgggcctgcgcaccgcgtgcgtgcggcgggtgctgccgctgctgtccgactccaccgtggacgccctgtgggcaccggagttccgcaccgcgccgccgccctccttccagctgagcggccccgaggaggcacccgcaccggtactcctcggcgaccgttcggaccccgacctgcgggtgaacctggcggccaccgagccggtgaccgagcgggccgccgaggccctgcgcgaactccaggcccacttcgacgccaccgccgtcacccaccgcctcctgcccggggagctggcgatcgtggacaaccgcgtcaccgtgcacggccgcaccgagttcaccccccgctacgacggcaccgaccgctggctccagcgcaccttcgtgctcaccgacctgcgccgctcgcgcgcgatgcggccggccgacggctacgtgctcggagcggccccgcagcccgcctga

SEQ ID NO: 4 - protein, 333 aa, Streptomyces coelicolor
  mutant of the natural asparagine oxygenase AsnO; Asn-241 is replaced by Asp-241
MAANAAGPASRYDVTLDQSDAELVEEIAWKLATQATGRPDDAEWVEAARNAWHAWPATLRRDLAGFRRDSGPDGAIVLRGLPVDSMGLPPTPRVNGSVQREASLGAAVLLMTACGLGDPGAFLPEKNGALVQDVVPVPGMEEFQGNAGSTLLTFHNENAFHEHRPDFVMLLCLRADPTGRAGLRTACVRRVLPLLSDSTVDALWAPEFRTAPPPSFQLSGPEEAPAPVLLGDRSDPDLRVNLAATEPVTERAAEALRELQAHFDATAVTHRLLPGELAIVDNRVTVHGRTEFTPRYDGTDRWLQRTFVLTDLRRSRAMRPADGYVLGAAPQPA

SEQ ID NO: 5 - DNA, 35 bp, Streptomyces coelicolor
  synthetic oligonucleotide primer (Operon) for directed mutagenesis of the AsnO wild type. Exchange of the GAC (wild type) for AAC (AsnO D241N).
ccccgacctgcgggtgaacctggcggccaccgagc

SEQ ID NO: 6 - DNA, 35 bp, Streptomyces coelicolor
  synthetic oligonucleotide primer (Operon) for directed mutagenesis of the AsnO-wild type. reverser primer exchange of the GTC (wild type) for GTT (AsnO D241N)
gctcggtggccgccaggttcacccgcaggtcgggg

SEQ ID NO: 7 - DNA, 1074 bp, Streptomyces coelicolor
  His7 fusion insert for the expression of AsnO D241N
atgaaacatcaccatcaccatcaccatagcgattacgacatccccactactgagaatctttattttcagggatccgctgcgaatgccgcgggaccggcgtcgcgctacgacgtgacgctggatcagtcggatgcggaactcgtcgaggagatcgcctggaaactcgccacgcaggcgaccgggcggcccgacgacgccgaatgggtcgaggcggccaggaacgcctggcacgcctggccggcgaccctgcgccgggacctcgccggattccgccgggactcgggaccggacggcgcgatcgtgctgcgcggtctgcccgtcgactccatggggctgccgccgaccccgcgggtcaacggctccgtgcagcgcgaggcctcgctgggcgccgccgtgctgctgatgaccgcctgcgggctcggcgaccccggcgcgttcctgccggagaagaacggcgccctcgtacaggacgtcgtccccgtgccgggcatggaggagttccagggcaacgccggttcgaccctgctgacgttccacaacgagaacgccttccacgagcaccgccccgacttcgtgatgctgctgtgcctgcgcgccgaccccacgggccgggcgggcctgcgcaccgcgtgcgtgcggcgggtgctgccgctgctgtccgactccaccgtggacgccctgtgggcaccggagttccgcaccgcgccgccgccctccttccagctgagcggccccgaggaggcacccgcaccggtactcctcggcgaccgttcggaccccgacctgcgggtgaacctggcggccaccgagccggtgaccgagcgggccgccgaggccctgcgcgaactccaggcccacttcgacgccaccgccgtcacccaccgcctcctgcccggggagctggcgatcgtggacaaccgcgtcaccgtgcacggccgcaccgagttcaccccccgctacgacggcaccgaccgctggctccagcgcaccttcgtgctcaccgacctgcgccgctcgcgcgcgatgcggccggccgacggctacgtgctcggagcggccccgcagcccgcctga

SEQ ID NO: 8 - protein, 357 aa, Streptomyces coelicolor
  sequence of the expressed protein after purification by means of Ni-NTA affinity chromatography
MKHHHHHHHSDYDIPTTENLYFQGSAANAAGPASRYDVTLDQSDAELVEEIAWKLATQATGRPDDAEWVEAARNAWHAWPATLRRDLAGFRRDSGPDGAIVLRGLPVDSMGLPPTPRVNGSVQREASLGAAVLLMTACGLGDPGAFLPEKNGALVQDVVPVPGMEEFQGNAGSTLLTFHNENAFHEHRPDFVMLLCLRADPTGRAGLRTACVRRVLPLLSDSTVDALWAPEFRTAPPPSFQLSGPEEAPAPVLLGDRSDPDLRVNLAATEPVTERAAEALRELQAHFDATAVTHRLLPGELAIVDNRVTVHGRTEFTPRYDGTDRWLQRTFVLTDLRRSRAMRPADGYVLGAAPQPA

SEQ ID NO: 9 - DNA, 1002 bp, Streptomyces coelicolor
  Base sequence of AsnO D241N without cloning artifacts; these are the His7 tag, a short linker region and the Tobacco Etch Virus Protease recognition site.
tccgctgcgaatgccgcgggaccggcgtcgcgctacgacgtgacgctggatcagtcggatgcggaactcgtcgaggagatcgcctggaaactcgccacgcaggcgaccgggcggcccgacgacgccgaatgggtcgaggcggccaggaacgcctggcacgcctggccggcgaccctgcgccgggacctcgccggattccgccgggactcgggaccggacggcgcgatcgtgctgcgcggtctgcccgtcgactccatggggctgccgccgaccccgcgggtcaacggctccgtgcagcgcgaggcctcgctgggcgccgccgtgctgctgatgaccgcctgcgggctcggcgaccccggcgcgttcctgccggagaagaacggcgccctcgtacaggacgtcgtccccgtgccgggcatggaggagttccagggcaacgccggttcgaccctgctgacgttccacaacgagaacgccttccacgagcaccgccccgacttcgtgatgctgctgtgcctgcgcgccgaccccacgggccgggcgggcctgcgcaccgcgtgcgtgcggcgggtgctgccgctgctgtccgactccaccgtggacgccctgtgggcaccggagttccgcaccgcgccgccgccctccttccagctgagcggccccgaggaggcacccgcaccggtactcctcggcgaccgttcggaccccgacctgcgggtgaacctggcggccaccgagccggtgaccgagcgggccgccgaggccctgcgcgaactccaggcccacttcgacgccaccgccgtcacccaccgcctcctgcccggggagctggcgatcgtggacaaccgcgtcaccgtgcacggccgcaccgagttcaccccccgctacgacggcaccgaccgctggctccagcgcaccttcgtgctcaccgacctgcgccgctcgcgcgcgatgcggccggccgacggctacgtgctcggagcggccccgcagcccgcctga

SEQ ID NO: 10 - protein, 333 aa, Streptomyces coelicolor
  mutant of the natural asparagine-oxygenase AsnO Amino acid sequence of AsnO D241N without cloning artifacts; these are the His7 tag, a short linker region and the Tobacco Etch Virus Protease recognition site.
SAANAAGPASRYDVTLDQSDAELVEEIAWKLATQATGRPDDAEWVEAARNAWHAWPATLRRDLAGFRRDSGPDGAIVLRGLPVDSMGLPPTPRVNGSVQREASLGAAVLLMTACGLGDPGAFLPEKNGALVQDVVPVPGMEEFQGNAGSTLLTFHNENAFHEHRPDFVMLLCLRADPTGRAGLRTACVRRVLPLLSDSTVDALWAPEFRTAPPPSFQLSGPEEAPAPVLLGDRSDPDLRVNLAATEPVTERAAEALRELQAHFDATAVTHRLLPGELAIVDNRVTVHGRTEFTPRYDGTDRWLQRTFVLTDLRRSRAMRPADGYVLGAAPQPA

SEQ ID NO: 11 - DNA, 918 bp, Streptomyces coelicolor
  directed DNA-mutagenesis of the natural asparagine- oxgenase AsnO Codon 721-723 (GAC in wild type for Asp) is replaced by AAC (for Asn).Bases 37-954
gacgtgacgctggatcagtcggatgcggaactcgtcgaggagatcgcctggaaactcgccacgcaggcgaccgggcggcccgacgacgccgaatgggtcgaggcggccaggaacgcctggcacgcctggccggcgaccctgcgccgggacctcgccggattccgccgggactcgggaccggacggcgcgatcgtgctgcgcggtctgcccgtcgactccatggggctgccgccgaccccgcgggtcaacggctccgtgcagcgcgaggcctcgctgggcgccgccgtgctgctgatgaccgcctgcgggctcggcgaccccggcgcgttcctgccggagaagaacggcgccctcgtacaggacgtcgtccccgtgccgggcatggaggagttccagggcaacgccggttcgaccctgctgacgttccacaacgagaacgccttccacgagcaccgccccgacttcgtgatgctgctgtgcctgcgcgccgaccccacgggccgggcgggcctgcgcaccgcgtgcgtgcggcgggtgctgccgctgctgtccgactccaccgtggacgccctgtgggcaccggagttccgcaccgcgccgccgccctccttccagctgagcggccccgaggaggcacccgcaccggtactcctcggcgaccgttcggaccccgacctgcgggtgaacctggcggccaccgagccggtgaccgagcgggccgccgaggccctgcgcgaactccaggcccacttcgacgccaccgccgtcacccaccgcctcctgcccggggagctggcgatcgtggacaaccgcgtcaccgtgcacggccgcaccgagttcaccccccgctacgacggcaccgaccgctggctccagcgcaccttcgtgctcaccgacctgcgccgctcgcgcgcgatg

SEQ ID NO: 12 - protein, 306 aa, Streptomyces coelicolor
  Mutant of the natural asparagine-oxygenase AsnO; Asp-241 is replaced by Asn-241; shown are amio acids 13-318
DVTLDQSDAELVEEIAWKLATQATGRPDDAEWVEAARNAWHAWPATLRRDLAGFRRDSGPDGAIVLRGLPVDSMGLPPTPRVNGSVQREASLGAAVLLMTACGLGDPGAFLPEKNGALVQDVVPVPGMEEFQGNAGSTLLTFHNENAFHEHRPDFVMLLCLRADPTGRAGLRTACVRRVLPLLSDSTVDALWAPEFRTAPPPSFQLSGPEEAPAPVLLGDRSDPDLRVNLAATEPVTERAAEALRELQAHFDATAVTHRLLPGELAIVDNRVTVHGRTEFTPRYDGTDRWLQRTFVLTDLRRSRAM

SEQ ID NO: 13 - DNA, 996 bp, Streptomyces coelicolor
  site-directed DNA-mutagenesis of the natural asparagin- oxygenase AsnO Codon 721-723 (GAC in wild type for Asp) is replaced by AAC (for Asn).Bases 4-999
gctgcgaatgccgcgggaccggcgtcgcgctacgacgtgacgctggatcagtcggatgcggaactcgtcgaggagatcgcctggaaactcgccacgcaggcgaccgggcggcccgacgacgccgaatgggtcgaggcggccaggaacgcctggcacgcctggccggcgaccctgcgccgggacctcgccggattccgccgggactcgggaccggacggcgcgatcgtgctgcgcggtctgcccgtcgactccatggggctgccgccgaccccgcgggtcaacggctccgtgcagcgcgaggcctcgctgggcgccgccgtgctgctgatgaccgcctgcgggctcggcgaccccggcgcgttcctgccggagaagaacggcgccctcgtacaggacgtcgtccccgtgccgggcatggaggagttccagggcaacgccggttcgaccctgctgacgttccacaacgagaacgccttccacgagcaccgccccgacttcgtgatgctgctgtgcctgcgcgccgaccccacgggccgggcgggcctgcgcaccgcgtgcgtgcggcgggtgctgccgctgctgtccgactccaccgtggacgccctgtgggcaccggagttccgcaccgcgccgccgccctccttccagctgagcggccccgaggaggcacccgcaccggtactcctcggcgaccgttcggaccccgacctgcgggtgaacctggcggccaccgagccggtgaccgagcgggccgccgaggccctgcgcgaactccaggcccacttcgacgccaccgccgtcacccaccgcctcctgcccggggagctggcgatcgtggacaaccgcgtcaccgtgcacggccgcaccgagttcaccccccgctacgacggcaccgaccgctggctccagcgcaccttcgtgctcaccgacctgcgccgctcgcgcgcgatgcggccggccgacggctacgtgctcggagcggccccgcagcccgcc

SEQ ID NO: 14 - protein, 332 aa, Streptomyces coelicolor
  mutant of the natural asparagine-oxygenase AsnO; Asp-241 is replaced by Asn-241; shown are amino acids 2-333
AANAAGPASRYDVTLDQSDAELVEEIAWKLATQATGRPDDAEWVEAARNAWHAWPATLRRDLAGFRRDSGPDGAIVLRGLPVDSMGLPPTPRVNGSVQREASLGAAVLLMTACGLGDPGAFLPEKNGALVQDVVPVPGMEEFQGNAGSTLLTFHNENAFHEHRPDFVMLLCLRADPTGRAGLRTACVRRVLPLLSDSTVDALWAPEFRTAPPPSFQLSGPEEAPAPVLLGDRSDPDLRVNLAATEPVTERAAEALRELQAHFDATAVTHRLLPGELAIVDNRVTVHGRTEFTPRYDGTDRWLQRTFVLTDLRRSRAMRPADGYVLGAAPQPA

SEQ ID NO: 15 - DNA, 1002 bp, Streptomyces coelicolor
  directed DNA-mutagenesis of the natural asparagine- oxygenase AsnO codon 721-723 (GAC in wild type for Asn) is replaced by AAT (fur Asn)
atggctgcgaatgccgcgggaccggcgtcgcgctacgacgtgacgctggatcagtcggatgcggaactcgtcgaggagatcgcctggaaactcgccacgcaggcgaccgggcggcccgacgacgccgaatgggtcgaggcggccaggaacgcctggcacgcctggccggcgaccctgcgccgggacctcgccggattccgccgggactcgggaccggacggcgcgatcgtgctgcgcggtctgcccgtcgactccatggggctgccgccgaccccgcgggtcaacggctccgtgcagcgcgaggcctcgctgggcgccgccgtgctgctgatgaccgcctgcgggctcggcgaccccggcgcgttcctgccggagaagaacggcgccctcgtacaggacgtcgtccccgtgccgggcatggaggagttccagggcaacgccggttcgaccctgctgacgttccacaacgagaacgccttccacgagcaccgccccgacttcgtgatgctgctgtgcctgcgcgccgaccccacgggccgggcgggcctgcgcaccgcgtgcgtgcggcgggtgctgccgctgctgtccgactccaccgtggacgccctgtgggcaccggagttccgcaccgcgccgccgccctccttccagctgagcggccccgaggaggcacccgcaccggtactcctcggcgaccgttcggaccccgacctgcgggtgaatctggcggccaccgagccggtgaccgagcgggccgccgaggccctgcgcgaactccaggcccacttcgacgccaccgccgtcacccaccgcctcctgcccggggagctggcgatcgtggacaaccgcgtcaccgtgcacggccgcaccgagttcaccccccgctacgacggcaccgaccgctggctccagcgcaccttcgtgctcaccgacctgcgccgctcgcgcgcgatgcggccggccgacggctacgtgctcggagcggccccgcagcccgcctga

SEQ ID NO: 16 - DNA, 996 bp, Streptomyces coelicolor
  directed DNA-mutagenesis of the natural asparagine- oxygenase AsnO codon 721-723 (GAC in wild type for Asn) is replaced by AAT (for Asn).Bases 4-999
gctgcgaatgccgcgggaccggcgtcgcgctacgacgtgacgctggatcagtcggatgcggaactcgtcgaggagatcgcctggaaactcgccacgcaggcgaccgggcggcccgacgacgccgaatgggtcgaggcggccaggaacgcctggcacgcctggccggcgaccctgcgccgggacctcgccggattccgccgggactcgggaccggacggcgcgatcgtgctgcgcggtctgcccgtcgactccatggggctgccgccgaccccgcgggtcaacggctccgtgcagcgcgaggcctcgctgggcgccgccgtgctgctgatgaccgcctgcgggctcggcgaccccggcgcgttcctgccggagaagaacggcgccctcgtacaggacgtcgtccccgtgccgggcatggaggagttccagggcaacgccggttcgaccctgctgacgttccacaacgagaacgccttccacgagcaccgccccgacttcgtgatgctgctgtgcctgcgcgccgaccccacgggccgggcgggcctgcgcaccgcgtgcgtgcggcgggtgctgccgctgctgtccgactccaccgtggacgccctgtgggcaccggagttccgcaccgcgccgccgccctccttccagctgagcggccccgaggaggcacccgcaccggtactcctcggcgaccgttcggaccccgacctgcgggtgaatctggcggccaccgagccggtgaccgagcgggccgccgaggccctgcgcgaactccaggcccacttcgacgccaccgccgtcacccaccgcctcctgcccggggagctggcgatcgtggacaaccgcgtcaccgtgcacggccgcaccgagttcaccccccgctacgacggcaccgaccgctggctccagcgcaccttcgtgctcaccgacctgcgccgctcgcgcgcgatgcggccggccgacggctacgtgctcggagcggccccgcagcccgcc

SEQ ID NO: 17 - DNA, 4803 bp, Artificial Sequence
  cloning vector pQTEV
ctcgagaaatcataaaaaatttatttgctttgtgagcggataacaattataatagattcaattgtgagcggataacaatttcacacagaattcattaaagaggagaaattaactatgaaacatcaccatcaccatcaccatagcgattacgacatccccactactgagaatctttattttcagggatccgggtcgactgttgatagatctcggtaccgcggccgctcgacctgcagccaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccctctagattacgtgcagtcgatgataagctgtcaaacatgagaattgtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcggaatttcgggcagcgttgggtcctggccacgggtgcgcatgatctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccacatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac